SEQUENCE LISTING (9 sequences)

SEQ ID NO: 1 - DNA, 42 bp, Artificial Sequence
  Description of Artificial Sequence synthetic DNA
ggaattcggccgacgccggtgaaactttcttgtccaccttgg

SEQ ID NO: 2 - DNA, 33 bp, Artificial Sequence
  Description of Artificial Sequence synthetic DNA
agctcaccggcgtccaccaagggcccatcggtc

SEQ ID NO: 3 - DNA, 35 bp, Artificial Sequence
  Description of Artificial Sequence synthetic DNA
ggtggacgccggtgagctcgagacggtgaccgtgg

SEQ ID NO: 4 - DNA, 19 bp, Artificial Sequence
  Description of Artificial Sequence synthetic DNA
ggaaacagctatgaccatg

SEQ ID NO: 5 - DNA, 68 bp, Artificial Sequence
  Description of Artificial Sequence synthetic DNA
gctctagaagcttataacttcgtataatgtatactatacgaagttatttcaaggagacagtcataatg

SEQ ID NO: 6 - DNA, 56 bp, Artificial Sequence
  Description of Artificial Sequence synthetic DNA
cagctctagataacttcgtataaggtatcctatacgaagttattaagactccttat

SEQ ID NO: 7 - DNA, 42 bp, Artificial Sequence
  Description of Artificial Sequence synthetic DNA
ggaattcgcggccgcaggcgcgccatctgtcttcatcttccc

SEQ ID NO: 8 - DNA, 46 bp, Artificial Sequence
  Description of Artificial Sequence synthetic DNA
ggaattcggcgccttggcgcgccttagcactctcccctgttgaagc

SEQ ID NO: 9 - DNA, 17 bp, Artificial Sequence
  Description of Artificial Sequence synthetic DNA
ctatgcggccccattca

(19) United States
(12) Patent Application Publication
(10) Pub. No.: US 2009/0311690 A1
(43) Pub. Date: Dec. 17, 2009

(54) VECTOR FOR SCREENING ANTIBODY

(75) Inventors: Hiroshi UEDA, Tokyo (JP); Jinhua Dong, Tokyo (JP); Masaki Ihara, Tokyo (JP); Masayuki Kawakami, Kanagawa (JP)

Correspondence Address: BIRCH STEWART KOLASCH & BIRCH, PO BOX 747, FALLS CHURCH, VA, 22040-0747, US

(73) Assignees: FUJIFILM Corporation, Tokyo (JP); THE UNIVERSITY OF TOKYO, Tokyo (JP)

(21) Appl. No.: 12/333,064

(22) Filed: Dec. 11, 2008

(30) Foreign Application Priority Data
Jun. 11, 2008 (JP) 2008-152656

(51) Int. Cl. C12Q 1/68; C12N 15/74; C12N 15/64

(52) U.S. Cl. 435/6; 435/320.1; 435/91.4

(57) ABSTRACT

It is an object of the present invention to provide a simple and efficient means capable of evaluating the VH/VL interaction without expressing/purifying VH and VL. The present invention provides a recombinant vector comprising: (i) a nucleotide sequence which can express a hetero-assembly composed of two types of fusion proteins wherein heavy chain variable region (VH) and light chain variable region (VL) of antibody are respectively fused with mutually associable first polypeptide and second polypeptide, by means of secretion, or in a form of a fusion protein tethered to a phage coat protein; and (ii) a restriction enzyme recognition sequence at two sites within, or in a vicinity of, a nucleotide sequence encoding said first polypeptide or second polypeptide.

### TECHNICAL FIELD

The present invention relates to a vector for screening antibodies suitable for noncompetitive immunoassay (open sandwich immunoassay) based on the antigen-dependent association between heavy chain variable region (VH) and light chain variable region (VL) of antibody, and a method for screening antibodies suitable for open sandwich immunoassay using such a vector.

### BACKGROUND ART

Open sandwich assay is a method in which: a VH region polypeptide and a VL region polypeptide of an antigen-specific antibody are prepared; either one of these polypeptides is labeled with a reporter molecule to make a labeled polypeptide, and the other polypeptide is immobilized onto a solid phase to make an immobilized polypeptide; and an antigen-containing sample and the labeled polypeptide are contacted with the solid phase, followed by quantification of the reporter molecule of the labeled polypeptide that has been bound to the immobilized polypeptide. The open sandwich assay is an immunoassay based on a phenomenon that the association constant between VH and VL is increased under the presence of an antigen. Therefore, the indispensable condition is that the VH/VL interaction is weak without an antigen but the association constant is greatly changed with the antigen. In order to accurately estimate the VH/VL interaction, VH and VL have to be separately expressed and purified. Therefore, this assay has required a large amount of time and labor so far.

A method called “split-Fv system” has been reported as a method to evaluate the VH/VL interaction without expressing/purifying VH and VL (PCT International Publication No. WO2004/016782). In this method, separate use of E. coli between those with and without an amber suppressor function against an amber (stop) codon in an expression vector, makes it possible to separately employ: a method in which VH and VL are respectively expressed as fusion proteins respectively tethered to phage coat proteins pVII and pIX; and a method in which either one of VH and VL is expressed as a fusion protein tethered to a phage coat protein pVII or pIX, and the other one of VH and VL is subjected to secretive expression. This method is capable of evaluating the affinity of the VH/VL complex for an antigen and the VH/VL interaction without the antigen, by changing the type of E. coli with a same vector.

However, this method has problems in that the use of two phage coat proteins leads to instability of the phage and consequent failure in the expression of VH/VL on the phage, and that the distance between VH/VL expressed as fusion proteins respectively tethered to coat proteins pVII and pIX is not enough for their interaction so that the affinity for the antigen is lowered.

In addition, as a method to evaluate the VH/VL interaction of the same concept, there is a reported method in which the affinity evaluation is carried out with a single-chain antibody (scFv) display phage, and then a sequence such as stop codon is recombined into a linker between VH/VL of scFv using a recombinase to achieve expression of VH and VL as separate components (for example, MBP-fusion VL and VH display phage) (Proceedings of 73rd Annual Meeting of the Society of Chemical Engineers, Japan, p. 366 (Efficient conversion of phage display antibody for open sandwich ELISA system with Cre recombinase). This method has several merits such that: (1) the scFv display can employ less toxic pIII among phage coat proteins; and (2) because of scFv, VH/VL is always present as a pair so that the panning can be stably performed. The recombination into the linker site can achieve insertion of a variety of sequences while retaining the direction, by using an enzyme having high sequence specificity such as Cre recombinase. For example, VH or VL can be secreted as a highly soluble and expressive MBP fusion protein. However, in this method, because two recombinase recognition sequences are present in the linker site, the linker length has to be elongated as very long as about 40 to 450 amino acids, although usual VH/VL linker of scFv is about 15 amino acids long. Accordingly, the display rate on the phage is lowered, which is problematic. In addition, an expensive recombinase is required for the recombination into the vector for evaluating the VH/VL interaction, and the recombination efficiency is not high, which have also been problematic.

### DISCLOSURE OF THE INVENTION

It is an object of the present invention to provide a simple and efficient means capable of evaluating the VH/VL interaction without expressing/purifying VH and VL, with a purpose of selecting VH and VL, the VH/VL interaction of which is weak without an antigen but the association constant of which is greatly changed with the antigen.

The inventors of the present invention have conducted intensive studies to solve the above problems. As a result, they have found that the VH/VL interaction can be simply and efficiently evaluated without expressing/purifying VH and VL, by inserting a restriction enzyme recognition sequence into two sites within, or in a vicinity of, a nucleotide sequence encoding a following first polypeptide or second polypeptide, in a recombinant vector comprising a nucleotide sequence which can express a hetero-assembly composed of two types of fusion proteins wherein heavy chain variable region (VH) and light chain variable region (VL) of antibody are respectively fused with mutually associable first polypeptide and second polypeptide, by means of secretion, or in a form of a fusion protein tethered to a phage coat protein. This has led to the completion of the present invention.

The present invention provides a recombinant vector comprising: (i) a nucleotide sequence which can express a hetero-assembly composed of two types of fusion proteins wherein heavy chain variable region (VH) and light chain variable region (VL) of antibody are respectively fused with mutually associable first polypeptide and second polypeptide, by means of secretion, or in a form of a fusion protein tethered to a phage coat protein; and (ii) a restriction enzyme recognition sequence at two sites within, or in a vicinity of, a nucleotide sequence encoding said first polypeptide or second polypeptide.

Preferably, the hetero-assembly is a Fab fragment which is a heterodimer which comprises: a fusion protein composed of heavy chain variable region (VH) and heavy chain constant region 1 (CH1) of antibody; and a fusion protein composed of light chain variable region (VL) and light chain constant region (CL) of antibody.

Preferably, the hetero-assembly is a F(ab′)2 fragment.

Preferably, the hetero-assembly is IgG.

Preferably, in the recombinant vector of the present invention, the respective nucleotide sequences are included in any order selected from the following (1) to (4), from the 5′ to 3′ direction:
(1) translational initiation sequence→VL gene sequence→CL gene sequence→stop codon→translational initiation sequence→VH gene sequence→restriction enzyme recognition sequence→CH1 gene sequence→restriction enzyme recognition sequence→phage coat protein sequence→stop codon;
(2) translational initiation sequence→VH gene sequence→restriction enzyme recognition sequence→CH1 gene sequence→restriction enzyme recognition sequence→phage coat protein sequence→stop codon→translational initiation sequence→VL gene sequence→CL gene sequence→stop codon;
(3) translational initiation sequence→VL gene sequence→restriction enzyme recognition sequence→CL gene sequence→restriction enzyme recognition sequence→stop codon→translational initiation sequence→VH gene sequence→CH1 gene sequence→phage coat protein sequence→stop codon; and
(4) translational initiation sequence→VH gene sequence→CH1 gene sequence→phage coat protein sequence→stop codon→translational initiation sequence→VL gene sequence→restriction enzyme recognition sequence→CL gene sequence→restriction enzyme recognition sequence→stop codon.

Preferably, the first polypeptide and the second polypeptide are respectively leucine zipper protein.

Preferably, the leucine zipper protein is Fos or Jun.

Preferably, the first polypeptide and the second polypeptide are respectively: a protease or an inactive mutant thereof, and a protease inhibitor; or vice versa.

The present invention further provides a method for producing a recombinant vector which is capable of extracellular secretion of a protein comprising either one of heavy chain variable region (VH) or light chain variable region (VL) of antibody and is capable of expression of a protein comprising the other one of heavy chain variable region (VH) or light chain variable region (VL) of antibody, by means of secretion, or in a form of a fusion protein tethered to a phage coat protein; which comprises (i) a step of digesting the recombinant vector of the present invention with a restriction enzyme that can cleave the restriction enzyme recognition sequence existing within, or in a vicinity of, the nucleotide sequence encoding the first polypeptide or second polypeptide, and (ii) a step of circularizing the vector obtained by the above step (i) to thereby construct a recombinant vector lacking the nucleotide sequence encoding the first polypeptide or the second polypeptide.

The present invention further provides a method for evaluating an interaction between heavy chain variable region (VH) and light chain variable region (VL) of antibody, which comprises effecting extracellular secretion of a protein comprising either one of heavy chain variable region (VH) or light chain variable region (VL) of antibody and expression of a protein comprising the other one of heavy chain variable region (VH) or light chain variable region (VL) of antibody by means of secretion or in a form of a fusion protein tethered to a phage coat protein, by the use of a recombinant vector produced by the method of the present invention.

Preferably, heavy chain variable region (VH) or light chain variable region (VL) of antibody with weak interaction is selected.

Preferably, a Fab fragment mixture having high affinity for a target antigen is selected among Fab fragment mixtures, and then heavy chain variable region (VH) or light chain variable region (VL) of antibody with weak interaction is selected.

The present invention is characterized in that an antibody to be used for assay is displayed on a phage coat protein, for example, as a Fab fragment (heterodimer of VH-CH1 and VL-CL). For the display of Fab on the phage: (i) firstly, a fusion protein of VH-CH1 and pIII is expressed; (ii) a heterodimer is formed through the interaction between separately expressed VL-CL and CH1-CL; and (iii) subsequently, the heterodimer is incorporated into the phage through mediation of pIII. In the present invention, a same restriction site is inserted into, for example, both ends of a CH1 gene in the vector. Then, a phagemid is extracted from phages selected as Fab display phages on the basis of their antigen-binding property, and is subjected to CH1 deletion and self-ligation through digestion with the restriction enzyme. The self-ligated product is then transformed into E. coli. By so doing, a culture supernatant containing both of the antigen-specific L chain and VH fragment display phage can be obtained. This culture supernatant is poured into a microplate immobilized with an L chain-specific binding protein, followed by evaluation of the binding amount of the phage to the plate with or without addition of an antigen. This evaluation enables efficient selection of antibodies suitable for open sandwich assay. The Fab fragment is similar to natural antibody and is known to have a high display rate. Thus, the method of the present invention is excellent in terms of evaluation of antigen specificity. In addition, transformation into a vector that can express VH and VL as separate components, can be achieved by simple gene manipulations such as one-off restriction enzyme digestion and self ligation, and therefore the method of the present invention is also excellent in terms of handiness. That is to say, according to the method of the present invention, antibodies suitable for use in open sandwich ELISA can be simply and efficiently selected according to the purpose.

### BRIEF DESCRIPTION OF THE DRAWINGS

FIG. 1 shows a production scheme of Fab/pDong1 (HyHEL-10).

FIG. 2 shows the results of phage-ELISA with Fab display phages.

FIG. 3 shows the results of open Sandwich ELISA.

FIG. 4 shows the results of panning with model libraries.

FIG. 5 shows the results of colony PCR with first round colonies obtained from model panning.

### BEST MODE FOR CARRYING OUT THE INVENTION

Hereinafter, the present invention will be more specifically described.

In Examples of this application exemplifying the present invention, on a filamentous phage coat protein is displayed a Fab fragment of an antibody to be used for assay, that is to say, a heterodimer in which a heavy chain variable region (VH) and a light chain variable region (VL) constituting the antigen recognition site are respectively linked to a heavy chain constant region (CH1) and a light chain constant region (CL). In particular, in the present invention, a highly specific restriction site is inserted into both ends of a gene sequence encoding the CH1 region in a phagemid vector. By so doing, the phagemid DNA is extracted from phages selected as Fab display phages on the basis of their antigen-binding property, and is subjected to self ligation through digestion with the restriction enzyme. The self-ligated product is again transformed into E. coli so as to prepare the phage, by which a culture supernatant containing both of the antigen-specific L chain and VH fragment display phage can be obtained. This culture supernatant is contacted with a microplate immobilized with an L chain-specific binding protein, followed by evaluation of the binding amount of the phage to the plate with or without addition of an antigen, using an enzyme-labeled anti-phage antibody. This evaluation enables the selection of VH and VL, the VH/VL interaction of which is weak without the antigen but the VH/VL association constant of which is increased with the antigen.

That is to say, the vector of the present invention is a recombinant vector comprising: (i) a nucleotide sequence which can express a hetero-assembly composed of two types of fusion proteins wherein heavy chain variable region (VH) and light chain variable region (VL) of antibody are respectively fused with mutually associable first polypeptide and second polypeptide, by means of secretion, or in a form of a fusion protein tethered to a phage coat protein; and (ii) a restriction enzyme recognition sequence at two sites within, or in a vicinity of, a nucleotide sequence encoding such a first polypeptide or second polypeptide.

Examples of the hetero-assembly can include Fab fragment of antibody, F(ab′)2 fragment of antibody, and an IgG antibody, and preferably Fab fragment of antibody. The Fab fragment means a heterodimer comprising: a fusion protein composed of heavy chain variable region (VH) and heavy chain constant region 1 (CH1) of antibody; and a fusion protein composed of light chain variable region (VL) and light chain constant region (CL) of antibody.

As to the structure of the recombinant vector of the present invention, when the hetero-assembly is Fab fragment of antibody, the respective nucleotide sequences can be included in any order selected from the following (1) to (4), from the 5′ to 3′ direction:

(1) translational initiation sequence→VL gene sequence→CL gene sequence→stop codon→translational initiation sequence→VH gene sequence→restriction enzyme recognition sequence→CH1 gene sequence→restriction enzyme recognition sequence→phage coat protein sequence→stop codon:

(2) translational initiation sequence→VH gene sequence→restriction enzyme recognition sequence→CH1 gene sequence→restriction enzyme recognition sequence→phage coat protein sequence→stop codon→translational initiation sequence→VL gene sequence→CL gene sequence→stop codon:

(3) translational initiation sequence→VL gene sequence→restriction enzyme recognition sequence→CL gene sequence→restriction enzyme recognition sequence→stop codon→translational initiation sequence→VH gene sequence→CH1 gene sequence→phage coat protein sequence→stop codon: and

(4) translational initiation sequence→VH gene sequence→CH1 gene sequence→phage coat protein sequence→stop codon→translational initiation sequence→VL gene sequence→restriction enzyme recognition sequence→CL gene sequence→restriction enzyme recognition sequence→stop codon.

In addition, as to the first polypeptide and the second polypeptide which are respectively fused to heavy chain variable region (VH) and light chain variable region (VL) of antibody may be any mutually associable polypeptides, and do not have to be constant region domains of antibody as mentioned above. Examples thereof may include: a combination of leucine zipper proteins (such as Fos and Jun); and a combination of a protease or an inactive mutant thereof, and a protease inhibitor.

In the present invention, a recombinant vector lacking the nucleotide sequence encoding the first polypeptide or second polypeptide can be constructed by digestion of the abovementioned recombinant vector of the present invention with a restriction enzyme that can cleave the restriction enzyme recognition sequence existing within, or in a vicinity of, the nucleotide sequence encoding the first polypeptide or second polypeptide, and subsequent circularization of the vector obtained by the digestion with the restriction enzyme. Such a recombinant vector lacking the nucleotide sequence encoding the first polypeptide or second polypeptide is capable of extracellular secretion of a protein comprising either one of the heavy chain variable region (VH) or light chain variable region (VL) of antibody, and expression of a protein comprising the other one of heavy chain variable region (VH) or light chain variable region (VL) of antibody, by means of secretion, or in a form of a fusion protein tethered to a phage coat protein.

There is no specific limitation as to the enzyme that can cleave the restriction enzyme recognition sequence existing within, or in a vicinity of, the nucleotide sequence encoding the first polypeptide or second polypeptide, although enzymes which are specific to eight or more nucleotides and produce same cohesive ends are preferably used. Examples thereof can include SgrAI, AscI, and NotI.

The term “translational initiation sequence” used herein is composed of a ribosomal binding site, an initiation codon, and a secretion signal sequence. Examples of the secretion signal sequence can include DNA sequences encoding an OmpA signal sequence, a pelB signal sequence, a gIII signal sequence, and the like.

Examples of the phage coat protein sequence can include a gIII protein, a gIII protein C-terminal domain (D3), and a gIX protein.

In the present invention, a phagemid vector is preferably used. Since a phagemid vector is a plasmid produced to include a part of filamentous phage genome, the phagemid vector is transformed into E. coli, and further infected with a helper phage. By so doing, coat proteins for particle formation are supplied, by which phages are provided in a form of a mixture of helper phage particles and phagemid particles. In addition, as a simpler method, a phage vector including necessary DNA sequences can also be used. In the case of a phage vector, phages can be directly provided through infection of the phage vector into E. coli, and there is no need of using a helper phage.

In the present invention, the abovementioned recombinant vector of the present invention can be used to effect extracellular secretion of a protein comprising either one of heavy chain variable region (VH) or light chain variable region (VL) of antibody, and expression of a protein comprising the other one of heavy chain variable region (VH) or light chain variable region (VL) of antibody, by means of secretion, or in a form of a fusion protein tethered to a phage coat protein. That is to say, in the present invention, digestion of the recombinant vector of the present invention with a restriction enzyme, and subsequent circularization of the vector obtained by the digestion with the restriction enzyme lead to a construction of a recombinant vector lacking the nucleotide sequence encoding the first polypeptide or second polypeptide. Such a vector is introduced into a host cell. Then, a protein comprising either one of VH fragment or VL fragment of the antibody's variable domain which has been secreted from the host cell, and a phage displaying the other one of the VH fragment or VL fragment of the antibody's variable domain, are collected. Thus collected protein comprising either one of the VH fragment or VL fragment of antibody's variable domain, and phage displaying the other one of the VH fragment or VL fragment of antibody's variable domain, are contacted with an antigen. Detection of a complex of the VH fragment, the VL fragment, and the antigen enables evaluation of the interaction between the VH polypeptide and the VL polypeptide. Preferably, it is possible to select heavy chain variable region (VH) or light chain variable region (VL) of antibody with weak interaction. In addition, a Fab fragment mixture having high affinity for a target antigen is selected among Fab fragment mixtures, and then heavy chain variable region (VH) or light chain variable region (VL) of antibody with weak interaction can be selected. The detection of a complex of the VH fragment, the VL fragment, and the antigen can be performed by open sandwich immunoassay that will be described below.

Protein antigens are generally assayed by a method called sandwich assay with use of two types of antibodies. Sandwich assay has several merits such as higher specificity and sensitivity, although two types of antibodies which can simultaneously bind to an antigen need to be prepared. However, small molecules having a molecular weight of 1000 or less are too small to be sandwiched by two antibodies. That is to say, a small molecule having a molecular weight of 1000 or less is a monovalent antigen having one antigen determinant, and thus is difficult to sandwich by two antibodies. Such small molecules are usually assayed by a method called competitive assay. However, the competitive assay has demerits such as difficulty in the condition setting, lower sensitivity, and requirements for considerable care and attention in the assay manipulations.

As a method which enables noncompetitive assay of small molecules without such demerits, the inventors of the present invention have reported an immunoassay approach called the open sandwich immunoassay. This assay is based on a principle that “a variable domain (antigen binding site) of antibody is unstable without an antigen, but is stabilized once an antigen is bound thereto”. An antibody is composed of two chains, namely an H chain and an L chain. Respective antigen binding sites thereof are called VH and VL, which constitute a minimum antigen recognition unit, namely the variable domain Fv. Recently, cloning of gene fragments encoding VH and VL can be easily performed with use of phage display method, and the like. However, the binding between VH and VL is noncovalent and is often unstable. In many cases, VH and VL are linked by a peptide to be used as a single-chain antibody (scFv).

The inventors of the present invention have found that some of such unstable Fv can be stabilized when an antigen is bound thereto, and the use of this phenomenon had realized simple, quick, and highly sensitive quantification of the antigen concentration. That is to say, they have found that quantification of phage or enzyme immobilized on a VL fragment-immobilized plate, after being contacted with a sample containing a phage- or alkaline phosphatase-conjugated VH fragment and an antigen, and subsequently washed once, showed a high correlation with the amount of the antigen (UEDA, H. et al. Nature Biotechnol. 14, 1714-1718 (1996)).

Further, the inventors of the present invention have developed a method for simple examination of available antibodies regarding the suitability for open sandwich assay (Aburatani, T. et al., Anal. Chem. 75, 4057-4064 (2003); Hiroshi Ueda, “A novel immunoassay capable of noncompetitive detection of small molecules”, Bio Medical Quick Review Nets No. 027 (2004); and Hiroshi Ueda, “Noncompetitive immunoassay of small molecules”, Seikagaku (Biochemistry), 76(7), 670-674 (2004))”. The use of this method (split-Fv system), which is similar to commercially available phage antibody system, enables handy examination of available hybridomas regarding both the antigen binding ability and the strength of VH/VL interaction of the antibody variable domain, by changing the phage-producing E. coli, and selection of more suitable antibodies.

In the present invention, the suitability for open sandwich assay can be quickly examined by contacting a protein comprising either one of the VH fragment or VL fragment of antibody variable domain and a phage displaying the other one of the VH fragment or VL fragment of antibody variable domain with an antigen at various concentrations, and detecting a complex of the VH fragment, the VL fragment and the antigen. The open sandwich assay preferably employs an antibody, the VH/VL interaction of which is weak without an antigen but the VH/VL interaction of which is strengthened with the antigen. With such antibody fragments suitable for open sandwich assay, the VL fragment (or VH fragment) immobilized on the carrier and the VH fragment (or VL fragment) displayed on the phage are rarely bound directly to each other without an antigen, and therefore the phage is hardly bound to the carrier. On the other hand, under the presence of the antigen, the VH fragment and the VL fragment are both well bound to the antigen, and the complex is stabilized, so that the phage can be bound to the carrier via the antigen Accordingly, quantification of the carrier-tethered phage with use of an anti-phage antibody enables selection of antibody fragments, the phage-binding amount of which largely varies depending on the presence of the antigen. If the interaction between the VH fragment and the VL fragment of the antibody variable domain is changed double or more under the presence of the antigen, such antibody fragments can be used for the present purpose.

It is possible to produce, for example, an assay kit as follows, with use of the antibody provided by the method of the present invention, the VH/VL interaction of which is weak in the absence of an antigen but the VH/VL interaction of which is strengthened in the presence of the antigen.

(1) The VL fragment is immobilized onto a tube or a microplate through biotin-avidin interaction or physical adsorption.

(2) A fusion protein of the VH fragment and a reporter enzyme (such as alkaline phosphatase) is produced and is contacted with the VL-immobilized solid phase together with a sample, for certain period of time.

(3) After washing, the activity of the immobilized enzyme is measured and is used as an indicator of the antigen concentration in the sample.

In addition, it is also possible to produce an assay kit as follows.

(1) The VH fragment and the VL fragment are labeled with two types of fluorescent dyes having mutually overlapping absorption/fluorescent spectrum (such as fluorescein and rhodamine).

(2) These are mixed with a sample, and left still for about 5 minutes, followed by exclusive excitation of the fluorescent dye having the shorter wavelength with exciting light. The measurement of fluorescence intensities derived from these two types of fluorescent dyes enables detection of fluorescence resonance energy transfer caused by the VH/VL association. The ratio between two fluorescence intensities is used as an indicator of the antigen concentration in the sample. This method enables measurement of the antigen concentration in a shorter time without washing operation, as compared to the former method.

Further, it is also possible to produce an assay kit as follows.

(1) The VH fragment and the VL fragment are expressed as fusion proteins with two types of enzyme fragments, each of which is not active per se, but the closely contacted pair of which shows improved activity (such as LacZΔα and LacZΔω), in E. coli, followed by purification.

(2) These two types of fusion proteins and a sample is mixed, and left still for a certain period of time. Then, a substrate (such as luminescent substrate Galacton Plus, Tropix, Bedford, Mass.) is mixed therein. The activity of the fusion protein complex is measured and is used as an indicator of the antigen concentration in the sample. This method enables measurement of the antigen concentration with much higher sensitivity without washing operation, as compared to the former two methods (Yokozeki et al., Anal. Chem. 74(11), 2500-2504, 2002).

The target of assay of the above method can include, firstly, specific proteins, peptides, various hormones, narcotic drugs, and therapeutic drugs in serum for clinical examinations. In addition, the target of assay of the present invention can also include dioxin, bisphenol A, nonyl phenol, and other presumably toxic chemical substances and agrochemicals in environmental water.

The present invention will be more specifically described in the following Examples. However, these Examples are not intended to limit the scope of the present invention.

### EXAMPLES

### Example 1

### Preparation of Fab Display Vector Fab/pDong

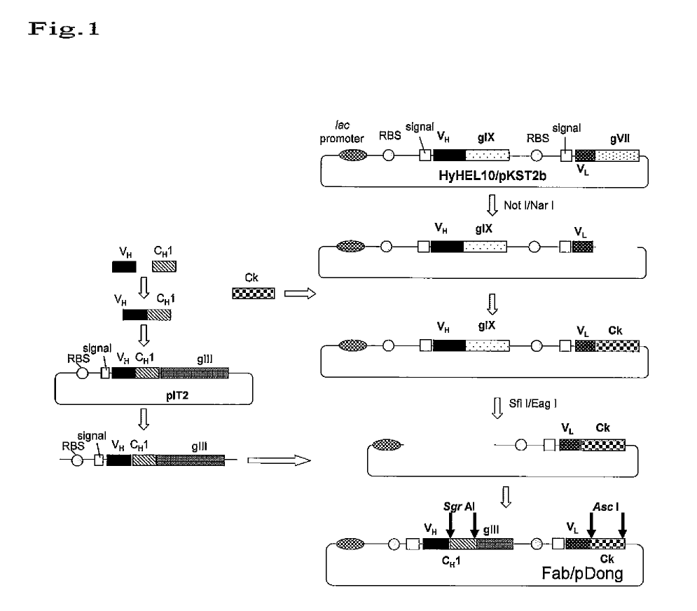

Fab-type antibody fragment display vector (Fab/pDong1) was produced as shown in FIG. 1. The primer sequences are shown in Table 1. CH1 and Ck of human Ig were used for the constant regions of the Fab fragments to be displayed, and VH and VL of a mouse antibody against hen egg-white lysozyme (HEL), HyHEL10, were used for the variable regions (V_{H}/V_{L}).

TABLE 1
Primers used for construction of Fab type antibody fragment display system

| | |
|---|---|
| Primer name | Sequence |

| | |
|---|---|
| hgCH1EagFor: | 5′ -GGAATTCGGCCGACGCCGGTGAAACTTTCTTGTCCACCTTGG-3′ |
| hgCH1SgrBack: | 5′ -AGCTCACCGGCGTCCACCAAGGGCCCATCGGTC-3′ |
| spH10VHSgrFor: | 5′ -GGTGGACGCCGGTGAGCTCGAGACGGTGACCGTGG-3′ |
| M13RV: | 5′ -GGAAACAGCTATGACCATG-3′ |
| FdLoxP511Back: | 5′ -GCTCTAGAAGCTTATAACTTCGTATAATGTATACTATACGAAGTTATTTCAAGGAGACAGTCATAATG-3′ |
| G3LoxP2272XbaFor: | 5′ -CAGCTCTAGATAACTTCGTATAAGGTATCCTATACGAAGTTATTAAGACTCCTTAT-3′ |
| hCkNotBack: | 5′ -GGAATTCGCGGCCGCAGGCGCGCCATCTGTCTTCATCTTCCC-3′ |
| hCkNarFor: | 5′ -GGAATTCGGCGCCTTGGCGCGCCTTAGCACTCTCCCCTGTTGAAGC-3′ |
| pHENseq: | 5′ -CTATGCGGCCCCATTCA-3′ |

The PCR reaction solution used in the following experiments was 50 μl of a reaction mixture containing 25 pmol of forward and reverse primers, about 50 ng of template, 4 μl of 0.25 mM dNTPs, 5 μl of 10×buffer for KOD Fx, 1 unit of KOD Fx (Toyobo, Osaka, Japan), and a balance of distilled water. The PCR reaction conditions were preheating at 94° C. for 2 minutes, and 30 cycles of 94° C. for 30 seconds, annealing for 30 seconds and extension reaction at 68° C. Annealing was carried out at the optimized temperature for each reaction. The duration of the extension reaction was 1 minute for amplification fragments of 1000 bp or shorter, with 1 minute increment per 1000 bp for amplification fragments of 1000 bp or longer. The PCR products and digested products with restriction enzymes were purified using Wizard® SV Gel and PCR Clean-Up System (Promega Co., Madison, Wis.).

Firstly, CH1 and Ck gene fragments were respectively amplified from plasmids inserted with human Ig gene (from Health Science Research Resources Bank) with use of respective primer pairs of hgCH1EagFor and hgCH1SgrBack, and hCkNarFor and hCkNotBack by PCR. The annealing temperature was 50° C. each case. The resultant Ck gene fragment was digested with NotI and NarI, purified, and mixed with pKST2b (HyHEL10) (Aburatani et al. Anal Chem. 2003, 75, 4057-4064) that had been similarly digested with NotI and NarI, and DNA Ligation high ver. 2 (TOYOBO CO., LTD, Osaka), followed by ligation reaction at 16° C. for 30 minutes. About 2 μl of ligation reaction solution was added to about 100 μl of E. coli XL10-Gold chemically competent cells to effect transformation. The transformant was cultured in LB agar medium containing 100 μg/ml Amp and 1% glucose at 37° C. overnight. A single colony was picked up and inoculated into 4 ml of LB medium containing 100 μg/ml Amp and 1% glucose overnight. The plasmid DNA was extracted from the culture product with use of Wizard® Plus Minipreps DNA Purification kit (Promega Co., Madison, Wis.), to obtain HyHEL10-Ck/pKST2b.

Next, in order to prepare the V_{H} gene fragment of HyHEL10, PCR reaction was performed at an annealing temperature of 50° C. using HyHEL10/pCANTAB as a template and spH10VHSgrFor and M13RV as a primer set. The resultant HyHEL10-V_{H} gene and CH1 fragment were mixed at approximately equal amounts, followed by 15 cycles of overlap PCR reaction without primers and 35 cycles of PCR reaction with hgCH1EagFor and M13RV primers (the annealing temperature was each 52° C.). The resultant VH-CH1 fusion gene was digested with SfiI and EagI, and mixed with phagemid vector pIT2 that had been similarly digested with SfiI and EagI, followed by ligation reaction and transformation in the same manner as the above to obtain VHCH1/pIT2.

A DNA fragment consisting of V_{H}-CH1-gIII was amplified by PCR with a template of VHCH1/pIT and primers of FdLoxP511Back and G3LoxP2272XbaFor (at an annealing temperature of 52° C.), digested with HindIII and XbaI, and mixed with HyHEL10-Ck/pKST2b that had been similarly digested with HindIII and XbaI, followed by ligation reaction in the same manner as the above and transformation into XL10-Gold to complete Fab/pDong1 (HyHEL-10). Sequencing was carried out to confirm that the resultant product actually had the sequence as designed.

### Example 2

### Confirmation of Antigen Binding Ability by Phage ELISA

The binding ability of HyHEL10-derived Fab antibody display phage that had been prepared so far by infection of E. coli TG-1 transformant of Fab/pDong1 with helper phage KM13, for the antigen and respective tagged antibodies, was confirmed by ELISA.

That is to say, E. coli TG-1 transformant of Fab/pDong1 was added to 10 ml of LB medium (100 μg/ml ampicillin and 1% glucose) and cultured at 37° C. until OD₆₀₀ reached about 0.4. Helper phage KM13 (2×10¹¹ pfu) was added thereto. The mixture was left still at 37° C. for 30 minutes, centrifuged (at 3,000 g for 10 minutes), and resuspended with 50 ml of LB medium (100 μg/ml ampicillin, 50 μg/ml kanamycin, and 0.1% glucose). The culture solution was transferred into a baffled Erlenmeyer flask, and incubated at 230 rpm at 30° C. After overnight incubation, bacteria were removed by centrifugation at 3,000 g for 30 minutes. About 40 ml of supernatant containing Fab display phage was added with 10 ml of PEG/NaCl (20% polyethylene glycol 6000 and 2.5 M NaCl), and was left still on ice for 1 hour. The solution was centrifuged at 3,300 g for 30 minutes. The supernatant was discarded and the pellet was dissolved with 1000 μl of TE, followed by centrifugation at 11,600 g for 10 minutes. After removing insoluble matters, the Fab display phage solution was collected. The titer was measured, and ELISA was performed in a plate immobilized with antigen in the following manner.

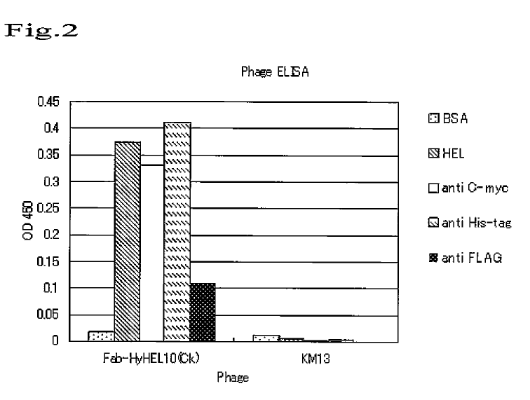

NaHCO₃ solution (pH 9.6) containing 10 μg/ml HEL, PBS solution containing 10 μg/ml BSA, 1 μg/ml anti-C-myc antibody, 1 μg/ml anti-His-tag antibody, and 1 μg/ml anti-FLAG antibody were dispensed in a Falcon 3912 microplate (Becton Dickinson and Company, Franklin Lakes, N.J.) at 100 μl each. The microplate was left still at 4° C. for 16 hours. After discarding solutions from the microplate, the microplate was blocked with 200 μl of PBS solution containing 2% skim milk (2% MPBS), at room temperature for 2 hours. Next, the microplate was washed with PBS solution containing 0.1% Tween 20 (PBST), added with 100 μl of MPBS and 10⁸ cfu of Fab display phage obtained from the above procedure, and left still at room temperature for 90 minutes. The microplate was washed with PBST. Then, in order to detect the immobilized Fab display phage, the microplate was added with 5000-fold diluted HRP/anti-M13 monoclonal conjugate (GE Healthcare UK Ltd., Amersham, UK) in MPBS and left still at room temperature for 1 hour. The microplate was then washed with PBST three times. Thereafter, a previously prepared enzyme reaction solution (50 ml of ELISA buffer, 500 μl of TMBZ (10 mg/ml in DMSO), and 10 μl of H₂O₂) was added to respective wells at 100 μl each to initiate the reaction. After incubation in dark for about 5 minutes, the reaction was stopped by addition of 50 μl of 3.2N H₂SO₄, and the absorbance was read at 450 nm (with reference at 655 nm) using a plate reader. As a result, as shown in FIG. 2, phages displaying Fab fragment of HyHEL 10 antibody showed high binding ability to HEL and respective V_{H}- and V_{L}-fused tags. Meanwhile, the culture supernatant containing KM13 as a negative control showed no significant signal.

### Example 3

### Panning of Model Libraries

In order to confirm that the Fab display phage of this system is applicable to panning of libraries, panning was carried out with model libraries.

The model libraries were produced by mixing 2.0×10⁸ cfu of HyHEL10 Fab display phage and 10¹² cfu of 13CG2 scFv display phage (phage displaying ScFv of anti-BS antibody) that had been produced in the same manner as the above. 3.6 ml of NaHCO₃ solution (pH 9.6) containing 10 μg/ml HEL was put into an immuno tube (Nalge Nunc International K.K., Rochester, N.Y.) and left still at 4° C. for 16 hours to immobilize antigens. After washing with PBS three times, blocking was performed with MPBS at room temperature for 2 hours. After washing with PBS three times, the tube was poured with 3.6 ml of solution containing 5.0×10¹¹ cfu of model library phage, and was rotated for 1 hour and left still for 1 hour at room temperature to immobilize these phages. After discarding the phage solution, the tube was washed with PBST twenty times, added with 500 μl of trypsin solution (1.0 mg/ml in PBS), and repeatedly inverted at room temperature for 10 minutes to effect elusion. 250 μl of the eluted phage solution was added to 1.75 ml of TG-1 in logarithmic growth phase, and left still at 37° C. for 30 minutes to infect these phages. 100-fold and 10,000-fold dilutions of this solution were spotted on a LB agar medium (100 μg/ml ampicillin and 1% glucose) and incubated at 37° C. overnight. The titer of the eluted phages was measured. The remaining solution was subcultured in 10 ml of LB liquid medium (100 μg/ml ampicillin and 1% glucose) at 37° C. until the OD₆₀₀ reached 0.4. Then, the solution was added with 5×10¹⁰ cfu of KM13 phage, and left still at 37° C. for 30 minutes to effect infection of the helper phage. After centrifugation at 3,000 g for 10 minutes, the supernatant was discarded, and the pellet was resuspended with 50 ml of LB liquid medium (100 μg/ml ampicillin, 50 μg/ml kanamycin, and 0.1% glucose), followed by incubation at 30° C. overnight. The culture solution was centrifuged at 3,300 g for 15 minutes. 40 ml of recovered supernatant was added with 10 ml of PEG/NaCl, and left still on ice for 1 hour, followed by centrifugation at 3,300 g for 30 minutes. PEG/NaCl was discarded. The pellet was suspended with 2 ml of TE, followed by centrifugation at 11,600 g for 10 minutes. E. coli debris was removed, and the supernatant was recovered.

### Example 4

### Polyclonal ELISA and Colony PCR

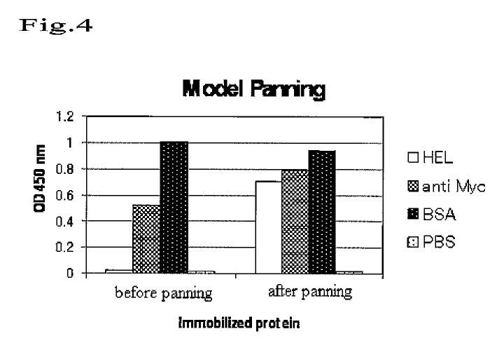

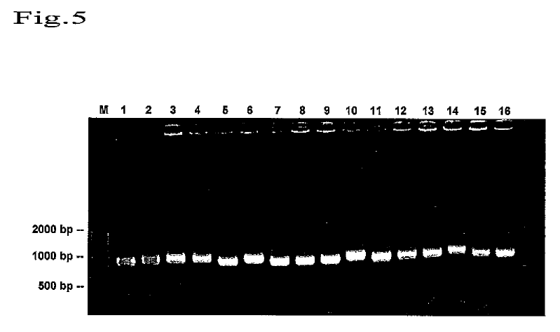

50 mM NaHCO₃ solution (pH 9.6) containing 10 μg/ml HEL, PBS solution containing 10 μg/ml BSA, PBS solution containing 1 μg/ml anti-C-myc antibody, and PBS solution were respectively dispensed in a Falcon 3912 microplate at 100 μl per well. The microplate was left still at 4° C. for 16 hours. Then, this plate was reacted with respective phages before and after panning at 5×10⁹ cfu per well, followed by ELISA in the same conditions as Example 2. As a result, as shown in FIG. 4, the signal to HEL remarkably increased after panning. Further, after the first round panning, colony PCR was performed to investigate the types of plasmids contained in the resultant clones. In the colony PCR, GoTaq Green Master Mix (Promega Co., Madison, Wis.) was added with 5 pmol of M13RV and pHENSEQ primers, and a balance of water to adjust the total volume of 10 μl. The solution was agitated with a toothpick adhered with a colony piece, and was subjected to PCR reaction at an annealing temperature of 50° C. Regarding the size of the amplification fragments calculated on the basis of sequence, the amplification fragment derived from Fab/pDong1 (HyHEL-10) is 852 bp while the amplification fragment derived from anti-BSA scFv display vector pIT2 (13CG2) is 902 bp. As a result of colony PCR, as shown in FIG. 5, the number of Fab/pDong1 (HyHEL-10) clones was ten and the number of pIT2 (13CG2) clones was five out of sixteen clones. That is to say, panning with HEL was able to concentrate the phages displaying HyHEL10 Fab fragments at about 10,000-fold.

### Example 5

### Transformation Into Vector for Open Sandwich ELISA (OS-ELISA), and Operation of OS-ELISA

About 50 ng of Fab/pDong1 (HyHEL-10) was digested with the restriction enzyme SgrAI using the attached reaction solution at 37° C. for 3 hours. After purification, DNA Ligation high ver. 2 was added to effect self ligation at 16° C. for 30 minutes. The ligation product was added to TG-1 chemical competent cells to effect transformation. The transformant was cultured in LB agar medium containing 100 μg/ml Amp and 1% glucose at 37° C. overnight. From the resultant colonies, a clone having a vector OS/pDong1 lacking the CH1 gene was selected through colony PCR. The colony PCR was carried out with hCkNarFor and M13RV primers in the same manner as the above.

The selected clone was cultured in 4 ml of 2YT medium containing 100 μg/ml Amp and 1% glucose (16 g/l bacto trypson (Difco), 10 g/l yeast extract (Difco), 5 g/l NaCl, and pH 7.6) (YTAG) at 37° C. until OD₆₀₀ reached about 0.4. Helper phage KM13 (2×10¹¹ pfu) was added thereto. The mixture was left still at 37° C. for 30 minutes, centrifuged (at 3,000 g for 10 minutes), and resuspended with 50 ml of 2YT medium containing 100 μg/ml ampicillin, 50 μg/ml kanamycin, and 0.1% glucose. The culture solution was transferred in a baffled Erlenmeyer flask, and incubated at 230 rpm at 30° C. After overnight incubation, bacteria were removed by centrifugation at 3,000 g for 30 minutes. About 40 ml of supernatant containing VH display phage and VL-Ck protein was recovered and subjected to OS-ELISA as follows.

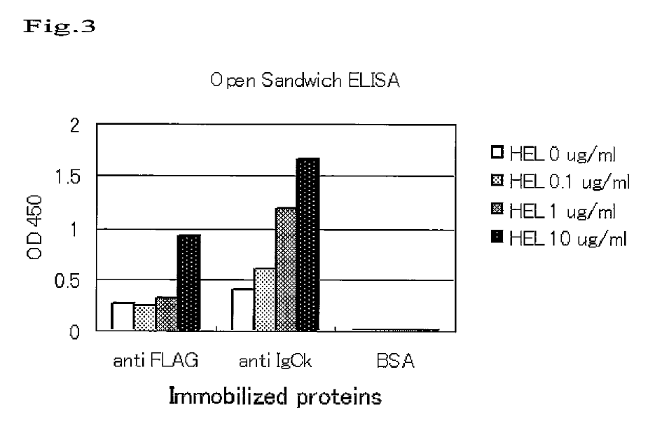

1 μg/ml anti-FLAG antibody and anti-IgK antibody were respectively dispensed in a Falcon 3912 microplate at 10 μl each. The microplate was left still at 4° C. for 16 hours. After discarding solutions from the microplate, the microplate was blocked with 200 μl of 2% MPBS at room temperature for 2 hours. Next, the microplate was washed with PBST solution, added with 100 μl of solution containing various concentrations of antigen (HEL) solution (0, 0.1, 1, and 10 μg/ml) and the phage supernatant (10 μl) prepared from the above procedure (in 1% MPBS), and left still at room temperature for 90 minutes. The microplate was washed with PBST. Then, in order to detect the immobilized VH display phage, the microplate was added with 5000-fold diluted HRP/anti-M13 monoclonal conjugate in MPBS and left still at room temperature for 1 hour. The microplate was then washed with PBST three times. Thereafter, an enzyme reaction solution was added to initiate the reaction. After incubation, the reaction was stopped with H₂SO₄, and the absorbance was read at 450 nm (with reference at 655 nm). As a result, as shown in FIG. 3, outstanding signal change was found in accordance with the increase in antigen concentration, showing that the display system is excellent for screening of antibodies suitable for OS-ELISA. Meanwhile, the plate immobilized with BSA as a negative control showed no signal change.

1. A recombinant vector comprising: (i) a nucleotide sequence which can express a hetero-assembly composed of two types of fusion proteins wherein heavy chain variable region (VH) and light chain variable region (VL) of antibody are respectively fused with mutually associable first polypeptide and second polypeptide, by means of secretion, or in a form of a fusion protein tethered to a phage coat protein; and (ii) a restriction enzyme recognition sequence at two sites within, or in a vicinity of, a nucleotide sequence encoding said first polypeptide or second polypeptide.

2. The recombinant vector according to claim 1, wherein the hetero-assembly is a Fab fragment which is a heterodimer which comprises: a fusion protein composed of heavy chain variable region (VH) and heavy chain constant region 1 (CH1) of antibody; and a fusion protein composed of light chain variable region (VL) and light chain constant region (CL) of antibody.

3. The recombinant vector according to claim 1, wherein the hetero-assembly is a F(ab′)2 fragment.

4. The recombinant vector according to claim 1, wherein the hetero-assembly is IgG.

5. The recombinant vector according to claim 2, wherein the respective nucleotide sequences are included in any order selected from the following (1) to (4), from the 5′ to 3′ direction:
(1) translational initiation sequence→VL gene sequence→CL gene sequence→stop codon→translational initiation sequence→VH gene sequence→restriction enzyme recognition sequence→CH1 gene sequence→restriction enzyme recognition sequence→phage coat protein sequence→stop codon;
(2) translational initiation sequence→VH gene sequence→restriction enzyme recognition sequence→CH1 gene sequence→restriction enzyme recognition sequence→phage coat protein sequence→stop codon→translational initiation sequence→VL gene sequence→CL gene sequence→stop codon;
(3) translational initiation sequence→VL gene sequence→restriction enzyme recognition sequence→CL gene sequence→restriction enzyme recognition sequence→stop codon→translational initiation sequence→VH gene sequence→CH1 gene sequence→phage coat protein sequence→stop codon; and
(4) translational initiation sequence→VH gene sequence→CH1 gene sequence→phage coat protein sequence→stop codon→translational initiation sequence→VL gene sequence→restriction enzyme recognition sequence→CL gene sequence→restriction enzyme recognition sequence→stop codon.

6. The recombinant vector according to claim 1, wherein the first polypeptide and the second polypeptide are respectively leucine zipper protein.

7. The recombinant vector according to claim 6, wherein the leucine zipper protein is Fos or Jun.

8. The recombinant vector according to claim 1, wherein the first polypeptide and the second polypeptide are respectively: a protease or an inactive mutant thereof, and a protease inhibitor; or vice versa.

9. A method for producing a recombinant vector which is capable of extracellular secretion of a protein comprising either one of heavy chain variable region (VH) or light chain variable region (VL) of antibody and is capable of expression of a protein comprising the other one of heavy chain variable region (VH) or light chain variable region (VL) of antibody, by means of secretion, or in a form of a fusion protein tethered to a phage coat protein; which comprises (i) a step of digesting the recombinant vector of claim 1 with a restriction enzyme that can cleave the restriction enzyme recognition sequence existing within, or in a vicinity of, the nucleotide sequence encoding the first polypeptide or second polypeptide, and (ii) a step of circularizing the vector obtained by the above step (i) to thereby construct a recombinant vector lacking the nucleotide sequence encoding the first polypeptide or the second polypeptide.

10. A method for evaluating an interaction between heavy chain variable region (VH) and light chain variable region (VL) of antibody, which comprises effecting extracellular secretion of a protein comprising either one of heavy chain variable region (VH) or light chain variable region (VL) of antibody and expression of a protein comprising the other one of heavy chain variable region (VH) or light chain variable region (VL) of antibody by means of secretion or in a form of a fusion protein tethered to a phage coat protein, by the use of a recombinant vector produced by the method of claim 9.

11. The method according to claim 10, wherein heavy chain variable region (VH) or light chain variable region (VL) of antibody with weak interaction is selected.

12. The method according to claim 11, wherein a Fab fragment mixture having high affinity for a target antigen is selected among Fab fragment mixtures, and then heavy chain variable region (VH) or light chain variable region (VL) of antibody with weak interaction is selected.
